(19) United States
(12) Patent Application Publication
(10) Pub. No.: US 2024/0132432 A1
(43) Pub. Date: Apr. 25, 2024

(54) FLUORINE-CONTAINING PILLARARENE

(71) Applicants: Kyoto University, Kyoto-shi (JP); DAIKIN INDUSTRIES, LTD., Osaka (JP)

(72) Inventors: Tomoki OGOSHI, Kyoto-shi (JP); Katsuto ONISHI, Kyoto-shi (JP); Shuhei YAMAGUCHI, Osaka (JP); Yuko SHIOTANI, Osaka (JP); Masaji KOMORI, Osaka (JP); Hirokazu AOYAMA, Osaka (JP); Yosuke KISHIKAWA, Osaka (JP); Akiyoshi YAMAUCHI, Osaka (JP)

(73) Assignees: Kyoto University, Kyoto-shi (JP); DAIKIN INDUSTRIES, LTD., Osaka (JP)

(21) Appl. No.: 18/542,922

(22) Filed: Dec. 18, 2023

Related U.S. Application Data
(63) Continuation of application No. PCT/JP22/24222, filed on Jun. 16, 2022.

(30) Foreign Application Priority Data
Jun. 17, 2021 (JP) 2021-101149

(51) Int. Cl. C07C 43/225; C07C 271/44

(52) U.S. Cl. CPC C07C 43/225; C07C 271/44; C07C 2602/48

(57) ABSTRACT

Provided is a compound represented by Formula (1A) below wherein the symbols are as defined in the description:

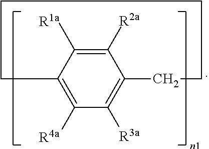

Also disclosed are a composition containing at least one of the compound and a formed article of the compound.

### CROSS REFERENCE TO RELATED APPLICATIONS

This application is a Rule 53(b) Continuation of International Application No. PCT/JP2022/024222 filed on Jun. 16, 2022, claiming priority based on Japanese Patent Application No. 2021-101149 filed on Jun. 17, 2021, the respective disclosures of which are incorporated herein by reference in their entirety.

### TECHNICAL FIELD

The present disclosure relates to a fluorine-containing pillararene.

### BACKGROUND ART

As a cyclic compound, a compound such as a pillararene, a crown ether, a cyclodextrin, a calixarene, or a cucurbituril is known. Among these, a pillararene, which has a symmetrical pillar structure, has attracted attention because of its various properties such as the ability to function as a host molecule that can incorporate a guest molecule (Patent Literature 1).

### CITATIION LIST

### Patent Literature

Patent Literature 1: JP 2015-221893 A

### SUMMARY

A compound represented by Formula (1A) below:

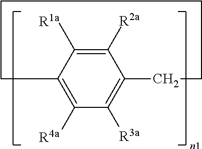

wherein R^{1a} is each independently at each occurrence a hydrogen atom or an organic group, R^{2a} is each independently at each occurrence a hydrogen atom or an organic group, R^{3a} is each independently at each occurrence a hydrogen atom or an organic group, R^{4a} is each independently at each occurrence a hydrogen atom or an organic group, provided that at least one of R^{1a}, R^{2a}, R^{3a}, and R^{4a} is an organic group having 2 or more carbon atoms and having a fluoroalkyl group or a fluorophenyl group, and n1 is an integer of 4 to 20.

### Advantageous Effect

According to the present disclosure, a fluorine-containing pillararene can be provided.

### DESCRIPTION OF EMBODIMENTS

As used herein, a “monovalent organic group” means a monovalent group containing carbon. The monovalent organic group is not limited, and can be a hydrocarbon group or a derivative thereof. A derivative of a hydrocarbon group means a group having one or more N, O, S, Si, amide, sulfonyl, siloxane, carbonyl, carbonyloxy, or the like at an end or in a molecular chain of the hydrocarbon group. When an “organic group” is simply shown, the organic group means a monovalent organic group.

As used herein, a “hydrocarbon group” means a group that contains carbon and hydrogen and that is obtained by removing one hydrogen atom from a hydrocarbon. The hydrocarbon group is not limited, and examples thereof include a C₁₋₂₀ hydrocarbon group optionally substituted with one or more substituents, such as an aliphatic hydrocarbon group or an aromatic hydrocarbon group. For example, the “aliphatic hydrocarbon group” may be linear, branched, or cyclic, and may be saturated or unsaturated. In addition, the hydrocarbon group may also include one or more ring structures.

As used herein, the substituent for the “hydrocarbon group” is not limited, and examples thereof include one or more groups selected from a halogen atom, a C₁₋₆ alkyl group, a C₂₋₆ alkenyl group, a C₂₋₆ alkynyl group, a C₃₋₁₀ cycloalkyl group, a C₃₋₁₀ unsaturated cycloalkyl group, a 5- to 10-membered heterocyclyl group, a 5- to 10-membered unsaturated heterocyclyl group, a C₆₋₁₀ aryl group, and a 5- to 10-membered heteroaryl group, optionally substituted with one or more halogen atoms.

In a first aspect, the present disclosure provides a compound represented by Formula (1A) below:

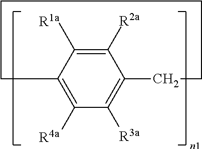

wherein R^{1a} is each independently at each occurrence a hydrogen atom or an organic group, R^{2a} is each independently at each occurrence a hydrogen atom or an organic group, R^{3a} is each independently at each occurrence a hydrogen atom or an organic group, R^{4a} each independently at each occurrence a hydrogen atom or an organic group, provided that at least one of R^{1a}, R^{2a}, R^{3a}, and R^{4a} is an organic group having 2 or more carbon atoms and having a fluoroalkyl group, and n1 is an integer of 4 to 20.

In Formula (1A) above, Rla is each independently at each occurrence a hydrogen atom or an organic group, R^{2a}, is each independently at each occurrence a hydrogen atom or an organic group, R^{3a}is each independently at each occurrence a hydrogen atom or an organic group, R^{4a} is each independently at each occurrence a hydrogen atom or an organic group, and at least one of R^{1a}, R^{2a}, R^{3a}, and R^{4a} is an organic group having 2 or more carbon atoms and having a fluoroalkyl group or a fluorophenyl group. In one embodiment, at least one of R^{1a}, R^{2a}, R^{3a}, and R^{4a} is an organic group having 2 or more carbon atoms and having a fluoroalkyl group.

The compound represented by Formula (1A) can have high miscibility, heat resistance, and water and oil repellency by having an organic group having 2 or more carbon atoms and having a fluoroalkyl group or a fluorophenyl group.

Examples of the organic group include a group including an alkyl group, an alkyloxy group, an alkyl ether group, a tosyl group, a triflate group, a phenyl group, or the like, optionally substituted with a substituent.

The substituent is not limited, and examples thereof include one or more groups selected from a halogen atom, a C₁₋₆ alkyl group, a C₂₋₆ alkenyl group, a C₂₋₆ alkynyl group, a C₃₋₁₀ cycloalkyl group, a C₃₋₁₀ unsaturated cycloalkyl group, a 5- to 10-membered heterocyclyl group, a 5- to 10-membered unsaturated heterocyclyl group, a C₆₋₁₀ aryl group, and a 5- to 10-membered heteroaryl group, optionally substituted with one or more halogen atoms, and a reactive functional group.

The reactive functional group is not limited, and examples thereof include a hydroxyl group, an amino group, a carboxyl group, a thiol group, an isocyanate group, a vinyl group, an acetylene group, a nitrile group, and an epoxy group.

The alkyl group is preferably a C₁₋₃₀ alkyl group. For example, the alkyl group may be linear or branched. In one embodiment, the C₁₋₃₀ alkyl group can be a C₁₋₂₀ alkyl group, preferably a C₁₋₁₀ alkyl group, and more preferably a C₁₋₃₀ alkyl group. In another embodiment, the C₁₋₃₀ alkyl group can be a C₁₇₋₃₀ alkyl group, preferably a C₁₈₋₃₀ alkyl group, and more preferably a C₂₆₋₃₀ alkyl group.

The alkyloxy group is typically a group represented by —O-R³¹ wherein R³¹ is an alkyl group.

The alkyl group of R³¹ is optionally substituted with any of the substituents described above, and preferably optionally substituted with a fluorine atom. The alkyl group is preferably a C₁₋₃₀ alkyl group optionally substituted with a fluorine atom. For example, the alkyl group may be linear or branched. In one embodiment, the C₁₋₃₀ alkyl group can be a C₁₋₂₀ alkyl group, preferably a C₁₋₁₀ alkyl group, and more preferably a C₁₋₃₀ alkyl group.

R³¹ is a group represented by preferably -R³²-R³³ wherein R³² is an unsubstituted C₁₋₃₀ alkylene group, preferably C₁₋₂₀ alkylene group, and more preferably C₁₋₁₀ alkylene group, and R³³ is a C₁₋₁₀ perfluoroalkyl group, and preferably a C₁₋₆ perfluoroalkyl group.

The alkyl ether group is a compound having one or more ethereal oxygen atoms in a molecular chain of the alkyl group. The alkyl ether group is typically a group represented by -R³⁶-R³⁷—(O-R³⁸)ₘ-R³⁹. In the above formula, R³⁶ is a single bond or an oxygen atom. R³⁷ is a single bond or a C₁₋₁₀ alkylene group. R³⁸ is a C₁₋₁₀ alkylene group. R³⁹ is a C₁₋₁₀ alkyl group optionally substituted with a fluorine atom.

In one embodiment, the organic group can be an alkyl group, an alkyloxy group, or an alkyl ether group, having an end substituted with a reactive functional group. The group is optionally further substituted with another substituent.

In one embodiment, R^{1a} and R^{3a} are each independently an organic group, and at least one of R^{1a} and R^{3a} is an organic group having 2 or more carbon atoms and having a fluoroalkyl group or a fluorophenyl group, preferably a fluoroalkyl group, or R^{2a} and R^{4a} are each independently an organic group, and at least one of R^{2a} and R^{4a} is an organic group having 2 or more carbon atoms and having a fluoroalkyl group or a fluorophenyl group, preferably a fluoroalkyl group.

In one embodiment, at least two of R^{1a}, R^{2a}, R^{3a}, and R^{4a} are each an organic group having 2 or more carbon atoms and having a fluoroalkyl group or a fluorophenyl group.

In one embodiment, at least two of R^{1a}, R^{2a}, R^{3a}, and R^{4a} are each an organic group having 2 or more carbon atoms and having a fluoroalkyl group.

In one embodiment, two of R^{1a}, R^{2a}, R^{3a}, and R^{4a} are each an organic group having 2 or more carbon atoms and having a fluoroalkyl group or a fluorophenyl group. In one embodiment, two of R^{1a}, R^{2a}, R^{3a}, and R^{4a} are each an organic group having 2 or more carbon atoms and having a fluoroalkyl group.

In one embodiment, R^{1a} and R^{3a} are each independently an organic group having 2 or more carbon atoms and having a fluoroalkyl group or a fluorophenyl group, preferably a fluoroalkyl group, or R^{2a} and R^{4a} are each independently an organic group having 2 or more carbon atoms and having a fluoroalkyl group or a fluorophenyl group, preferably a fluoroalkyl group.

In one embodiment, R^{2a} and R^{4a} are each a hydrogen atom, or R^{1a} and R^{3a} are each a hydrogen atom.

In a preferable embodiment, R^{1a} and R^{3a} are each independently an organic group having 2 or more carbon atoms and having a fluoroalkyl group or a fluorophenyl group, preferably a fluoroalkyl group, and R^{2a} and R^{4a} are each a hydrogen atom, or R^{2a} and R^{4a} are each independently an organic group having 2 or more carbon atoms and having a fluoroalkyl group or a fluorophenyl group, preferably a fluoroalkyl group, and R^{1a} and R^{3a} are each a hydrogen atom.

In a more preferable embodiment, R^{1a} and R^{3a} are each an organic group having 2 or more carbon atoms and having a fluoroalkyl group or a fluorophenyl group, preferably a fluoroalkyl group, and Rea and R^{4a} are each a hydrogen atom, or R^{2a} and R^{4a} are each an organic group having 2 or more carbon atoms and having a fluoroalkyl group or a fluorophenyl group, preferably a fluoroalkyl group, and R^{1a} and R^{3a} are each a hydrogen atom.

The fluoroalkyl group is preferably a C₁₋₁₀ fluoroalkyl group, and more preferably a C₁₋₆ fluoroalkyl group. For example, the fluoroalkyl group may be linear or branched, and is preferably linear. The fluoroalkyl group is preferably a perfluoroalkyl group.

For example, the fluorophenyl group may be a phenyl group substituted with 1 to 5 fluorine atoms, and is preferably a phenyl group substituted with 5 fluorine atoms.

In one embodiment, the organic group having 2 or more carbon atoms having a fluoroalkyl group is a group represented by the following formula: —(Oₚ₁-R^{11a}_{q1})-Rf^{a} wherein R^{11a} is each independently at each occurrence a C₁₋₁₀ alkylene group or —CONH—, R^{11a} is a C₁₋₁₀ fluoroalkyl group, p1 is an integer of 0 to 2, q1 is an integer of 0 to 3, and in (Oₚ₁-R^{11a}_{q1}), occurrence order of O and R^{11a} is not limited.

The C₁₋₁₀ alkylene group in Rila is preferably a C₁₋₆ alkylene group, and more preferably a C₂₋₆ alkylene group. For example, the alkylene group may be linear or branched, and is preferably linear.

In one embodiment, R^{11a} is each independently at each occurrence a C₁₋₁₀ alkylene group.

In another embodiment, R^{11a} is each independently at each occurrence —CONH—.

The C₁₋₁₀ fluoroalkyl group in Rf^{a} is preferably a C₁₋₆ fluoroalkyl group. For example, the fluoroalkyl group may be linear or branched, and is preferably linear. The fluoroalkyl group is preferably a perfluoroalkyl group.

p1 is an integer of 0 to 2, and is preferably 1 or 2. In one embodiment, p1 is 1. In another embodiment, p1 is 2.

q1 is an integer of 0 to 3, and preferably an integer of 1 to 3, and is more preferably 1 or 2. In one embodiment, q1 is 1. In another embodiment, q1 is 2.

In a preferable embodiment, the organic group having 2 or more carbon atoms and having a fluoroalkyl group is a group represented by —O-R^{11a}-(O-R^{11a})ᵣ₁-Rf^{a} wherein R^{11a} is each independently a C₁₋₁₀ alkylene group or —CONH—, Rf^{a} is a C₁₋₁₀ fluoroalkyl group, and r1 is 0 or 1.

In one a more preferable embodiment, the organic group having 2 or more carbon atoms and having a fluoroalkyl group is a group represented by —O-R^{11a}-(O-R^{11a})ᵣ₁-Rf^{a} wherein R^{11a} is each independently a C₁₋₁₀ alkylene group or —CONH—, Rf^{a} is a C₁₋₁₀ perfluoroalkyl group, and r1 is 0 or 1.

In one aspect, the organic group having 2 or more carbon atoms and having a fluorophenyl group is a group represented by the following formula: —(Oₚ₃-R^{11p}_{q3})-Rf^{p} wherein R^{11P} is each independently at each occurrence a C₁₋₁₀ alkylene group, preferably a C₁₋₆ alkylene group, and more preferably a C₁₋₃ alkylene group, Rf^{P} is a phenyl group substituted with 1 to 5, preferably 5, fluorine atoms, p3 is an integer of 0 to 2, q3 is an integer of 0 to 3, and in (Oₚ₃-R^{11p}_{q3}), occurrence order of O and R^{11p} is not limited.

The C₁₋₁₀ alkylene group in R^{11p} is preferably a C₁₋₆ alkylene group, and more preferably a C₁₋₆ alkylene group. For example, the alkylene group may be linear or branched, and is preferably linear.

p3 is an integer of 0 to 2, and is preferably 1 or 2. In one embodiment, p1 is 1. In another embodiment, p1 is 2.

q3 is an integer of 0 to 3, and preferably an integer of 1 to 3, and is more preferably 1 or 2. In one embodiment, q1 is 1. In another embodiment, q1 is 2.

In a preferable embodiment, the organic group having 2 or more carbon atoms and having a fluorophenyl group is a group represented by the following formula: —O-R^{11p}-Rf^{p} R^{11p} is each independently at each occurrence a C₁₋₁₀ alkylene group, preferably a C₁₋₆ alkylene group, and more preferably a C₁₋₃ alkylene group, and Rf^{p} is a phenyl group substituted with 1 to 5, preferably 5, fluorine atoms.

n1 is an integer of 4 to 20, and preferably an integer of 4 to 6, and is more preferably 5 or 6.

In a particularly preferable embodiment, R^{1a} and R^{3a} are each —O-R^{11a}-(O-R^{11a})ᵣ₁-Rf^{a}, and R^{2a} and R^{4a} are each a hydrogen atom, or R^{2a} and R^{4a} are each —O-R^{11a}-(O-R^{11a})ᵣ₁-Rf^{a}, and R^{1a} and R^{3a} are each a hydrogen atom, R^{11a} is each independently a C₁₋₁₀ alkylene group, and preferably a C₁₋₆ alkylene group, Rf^{a} is a C₁₋₁₀ perfluoroalkyl group, and preferably a C₁₋₆ perfluoroalkyl group, r1 is 0 or 1, and n1 is an integer of 4 to 6, and is preferably 5 or 6.

In another particularly preferable embodiment, R^{1a} and R^{3a} are each —O-R^{11p}-Rf^{p}, and R^{2a} and R^{4a} are each a hydrogen atom, or R^{2a} and R⁴ a are —O-R^{11p}-Rf^{p}, and R^{2a} and R^{2a} are each a hydrogen atom, R^{11p}is each independently a C₁₋₆ alkylene group, and preferably a C₁₋₃ alkylene group, Rf^{p} is a phenyl group substituted with 1 to 5, preferably 5, fluorine atoms, and n1 is an integer of 4 to 6, and is preferably 5 or 6.

The molecular weight of the compound represented by Formula (1A) above can be preferably 500 to 10,000, and more preferably 1,000 to 5,000.

When the compound represented by Formula (1A) above is formed into a film, the static contact angle of the resulting film with water is 80° or more, and preferably 90° or more.

The compound represented by Formula (1A) above can be obtained by converting a substituent of a pillar[n]arene to an organic group having 2 or more carbon atoms and having a fluoroalkyl group. Typically, the compound represented by Formula (1A) above can be obtained by introducing a hydroxyl group at a predetermined position of a pillar[n]arene and reacting the hydroxyl group with a tosylate, a halide, or the like of an organic group having 2 or more carbon atoms and having a fluoroalkyl group.

In a second aspect, the present disclosure provides a compound represented by Formula (1B) below:

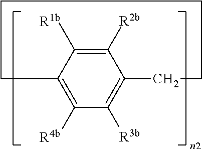

where R^{1b} is each independently at each occurrence a hydrogen atom or an organic group, R^{2b} is each independently at each occurrence a hydrogen atom or an organic group, R^{3b} is each independently at each occurrence a hydrogen atom or an organic group, and R^{4b} is each independently at each occurrence a hydrogen atom or an organic group, provided that at least one of R^{1b}, R^{2b}, R^{3b}, and R^{4b} is an organic group having a fluorine atom, and the organic group having a fluorine atom has an SP value of 8.0 or less, and n2 is an integer of 4 to 20.

As used herein, the SP value is a solubility parameter and serves as a measure of solubility. The SP value is a value unique to a substance; and a larger numerical value thereof shows higher polarity, and a smaller numerical value thereof shows lower polarity. The SP value is the value δ(cal/cm³)^{1/2} calculated from the following expression after determining the evaporation energy (Δei) and the molar volume (Lvi) by using the Fedors method (R.F. Fedors. Polyme. Eng. Sic., 14, 147(1974)).

δ=(ΣΔei/ΣΔvi)^{1/2}

In Formula (1B) above, R^{1b} is each independently at each occurrence a hydrogen atom or an organic group, R^{2b} is each independently at each occurrence a hydrogen atom or an organic group, R^{3b} is each independently at each occurrence a hydrogen atom or an organic group, R^{4b} is each independently at each occurrence a hydrogen atom or an organic group, at least one of R^{1b}, R^{2b}, R^{3b}, and R^{4b} is an organic group having a fluorine atom, and the organic group having a fluorine atom has an SP value of 8.0 or less.

The compound represented by Formula (1B) can have high miscibility, heat resistance, and water and oil repellency by having an organic group having a fluorine atom and having an SP value of 8.0 or less.

The organic group is defined as described for the organic group in Formula (1A) above.

In one embodiment, R^{1b} and R^{3b} are each independently an organic group, and at least one of R^{1b} and R^{3b} is an organic group having a fluorine atom and having an SP value of 8.0 or less, or R^{2b} and R^{4b} are each independently an organic group, and at least one of R^{2b} and R^{4b} is an organic group having a fluorine atom and having an SP value of 8.0 or less.

In one embodiment, at least two of R^{1b}, R^{2b}, R^{3b}, and R^{4b} are each an organic group having a fluorine atom and having an SP value of 8.0 or less.

In one embodiment, two of R^{1b}, R^{2b}, R^{3b}, and R^{4b} are each an organic group having a fluorine atom and having an SP value of 8.0 or less.

R^{1b} and R^{3b} are each independently an organic group having a fluorine atom and having an SP value of 8.0 or less, or R^{2b} and R^{4b} are each independently an organic group having a fluorine atom and having an SP value of 8.0 or less.

In one embodiment, R^{2b} and R^{4b} are each a hydrogen atom, or R^{1b} and R^{3b} are each a hydrogen atom.

In a preferable embodiment, R^{1b} and R^{3b} are each independently an organic group having a fluorine atom and having an SP value of 8.0 or less, and R^{2b} and R^{4b} are each a hydrogen atom, or R^{2b} and R 4 b are each independently an organic group having a fluorine atom and having an SP value of 8.0 or less, and R^{2b} and R 4 b are each a hydrogen atom.

In a more preferable embodiment, R ib and R^{3b} are each an organic group having a fluorine atom and having an SP value of 8.0 or less, and R^{2b} and R^{4b} are each a hydrogen atom, or R^{2b} and R^{4b} are each an organic group having a fluorine atom and having an SP value of 8.0 or less, and Rib and R^{3b} are each a hydrogen atom.

The SP value of the organic group having a fluorine atom is preferably 4.0 to 7.5, and more preferably 6.0 to 7.5.

In a preferable embodiment, the organic group having a fluorine atom has a Ci-io fluoroalkyl group. The fluoroalkyl group is preferably a C₁₋₆ fluoroalkyl group. For example, the fluoroalkyl group may be linear or branched, and is preferably linear. The fluoroalkyl group is preferably a perfluoroalkyl group.

In another preferable embodiment, the organic group having a fluorine atom has a fluorophenyl group. The fluorophenyl group is a phenyl group substituted with 1 to 5, preferably 5, fluorine atoms.

In one embodiment, the organic group having a fluorine atom is a group represented by the following formula: —(Oₚ₂R^{11b}_{q2})-Rf^{b} wherein R^{11b} is each independently at each occurrence a C₁₋₁₀ alkylene group, Rf^{b} is a C₁₋₁₀ fluoroalkyl group, p2 is an integer of 0 to 2, q2 is an integer of 0 to 3, and in (Oₚ₂-R^{11b}_{q2}), occurrence order of O and R^{11b} is not limited.

In one embodiment, the organic group having 2 or more carbon atoms having a fluorophenyl group is a group represented by the following formula: —(Oₚ₁-R^{11a} _{q1})-Rf^{p} wherein R^{11a} is each independently at each occurrence a C₁₋₁₀ alkylene group, Rf^{p} is a phenyl group substituted with 1 to 5, preferably 5, fluorine atoms, p1 is an integer of 0 to 2, q1 is an integer of 0 to 3, and in (Oₚ₁-R^{11a}_{q1}), occurrence order of O and R^{11a} is not limited.

The C₁₋₁₀ alkylene group in R^{11b} is preferably a C₁₋₆ alkylene group, and more preferably a C₂₋₆ alkylene group. For example, the alkylene group may be linear or branched, and is preferably linear.

The C₁₋₁₀ fluoroalkyl group in Rfb is preferably a C₁₋₆ fluoroalkyl group. For example, the fluoroalkyl group may be linear or branched, and is preferably linear. The fluoroalkyl group is preferably a perfluoroalkyl group.

p2 is an integer of 0 to 2, and is preferably 1 or 2. In one embodiment, p2 is 1. In another embodiment, p2 is 2.

q2 is an integer of 0 to 3, and preferably an integer of 1 to 3, and is more preferably 1 or 2. In one embodiment, q2 is 1. In another embodiment, q2 is 2.

In a preferable embodiment, the organic group having a fluorine atom is a group represented by —O-R^{11b})ᵣ₂-Rf^{b} wherein R^{11b} is each independently a C₁₋₁₀ alkylene group, Rf^{b} is a C₁₋₁₀ fluoroalkyl group, and r2 is 0 or 1.

In a more preferable embodiment, the organic group having a fluorine atom is a group represented by —O-R^{11b}-(O-R^{11b}b)ᵣ₂-Rf^{b} wherein R^{11b} is each independently a C₁₋₁₀ alkylene group, Rf^{b} is a C₁₋₁₀ fluoroalkyl group, and r2 is 0 or 1.

n2 is an integer of 4 to 20, and preferably an integer of 4 to 6, and is more preferably 5 or 6.

In a particularly preferable embodiment, R^{1b} and R^{3b} are each —O-R^{11b}-(O-R^{11b})ᵣ₂-Rf^{b}, and R^{2b} and R^{4b} are each a hydrogen atom, or R^{2b} and R^{4b} are each —O-R^{11b}-(O-R^{11b})ᵣ₂-Rf^{b}, and R^{1b} and R^{3b} are each a hydrogen atom, R^{11b} is each independently a C₁₋₁₀ alkylene group, and preferably a C₁₋₆ alkylene group, Rf^{b} is a C₁₋₁₀ perfluoroalkyl group, and preferably a C₁₋₆ perfluoroalkyl group, r2 is 0 or 1, and n2 is an integer of 4 to 6, and is preferably 5 or 6.

The molecular weight of the compound represented by Formula (1B) above can be preferably 500 to 10,000, and more preferably 1,000 to 5,000.

When the compound represented by Formula (1B) above is formed into a film, the static contact angle of the resulting film with water is 80° or more, and preferably 90° or more.

The compound represented by Formula (1B) above can be produced by the same method as for the compound represented by Formula (1A) above. For example, the compound represented by Formula (1B) above can be obtained by introducing a hydroxyl group at a predetermined position of a pillar[n]arene and reacting the hydroxyl group with a tosylate, a halide, or the like of an organic group having a fluorine atom.

In a third aspect, the present disclosure provides a compound represented by Formula (1C) below:

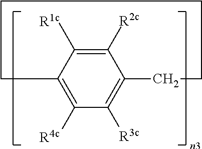

wherein R^{1c} is each independently at each occurrence a hydrogen atom or an organic group, R^{2c} is each independently at each occurrence a hydrogen atom or an organic group, R^{3c} is each independently at each occurrence a hydrogen atom or an organic group, R^{4c} is each independently at each occurrence a hydrogen atom or an organic group, provided that at least one of R^{1c}, R^{2c}, R^{3c}, and R^{4c} is an organic group having a long-chain alkyl group having 17 or more carbon atoms, and n3 is an integer of 4 to 20.

In Formula (1C) above, Rio is each independently at each occurrence a hydrogen atom or an organic group, R^{2c} is each independently at each occurrence a hydrogen atom or an organic group, R^{3c} is each independently at each occurrence a hydrogen atom or an organic group, R^{4c} is each independently at each occurrence a hydrogen atom or an organic group, and at least one of R^{1c}, R^{2c}, R^{3c}, and R^{4c} is an organic group having a long-chain alkyl group having 17 or more carbon atoms.

The compound represented by Formula (1C) can have high miscibility, heat resistance, and water repellency by having an organic group having a long-chain alkyl group having 17 or more carbon atoms.

The organic group is defined as described for the organic group in Formula (1A) above.

The number of carbon atoms in the long-chain alkyl group is preferably 17 to 30, more preferably 18 to 30, and further preferably 26 to 30.

The organic group having a long-chain alkyl group is preferably a group represented by —O-R^{12c} wherein R^{12c} is a long-chain alkyl group having 17 or more carbon atoms, preferably a C₁₇₋₃₀ alkyl group, and preferably a C₁₈₋₃₀ alkyl group or a C₂₆₋₃₀ alkyl group.

For example, the long-chain alkyl group may be linear or branched, and is preferably linear.

In one embodiment, R^{1c} and R^{3c} are each independently an organic group, and at least one of R^{1c} and R^{3c} is an organic group having a long-chain alkyl group having 17 or more carbon atoms, or R^{2c} and R^{4c} are each independently an organic group, and at least one of R^{2c} and R^{4c} is an organic group having a long-chain alkyl group having 17 or more carbon atoms.

In one embodiment, at least two of R^{1c}, R^{2c}, R^{3c}, and R^{4c} are each an organic group having a long-chain alkyl group having 17 or more carbon atoms.

In one embodiment, two of R^{1c}, R^{2c}, R^{3c}, and R^{4c} are each an organic group having a long-chain alkyl group having 17 or more carbon atoms.

In one embodiment, R^{1c} and R^{3c} are each independently an organic group having a long-chain alkyl group having 17 or more carbon atoms, and preferably —O-R^{12c} , or R^{2c} and R^{4c} are each independently an organic group having a long-chain alkyl group having 17 or more carbon atoms, and preferably —O-R^{12c} .

In one embodiment, R^{2c} and R^{4c} are each a hydrogen atom, or R^{1c} and R^{3c} are each a hydrogen atom.

In a preferable embodiment, R^{1c} and R^{3c} are each independently an organic group having a long-chain alkyl group having 17 or more carbon atoms, and R^{2c} and R^{4c} are each a hydrogen atom, or R^{2c} and R^{4c} are each independently an organic group having a long-chain alkyl group having 17 or more carbon atoms, and R^{3c} and R^{3c} are each a hydrogen atom.

In a more preferable embodiment, R^{3c} and R^{3c} are each an organic group having a long-chain alkyl group having 17 or more carbon atoms, and R^{2c} and R^{4c} are each a hydrogen atom, or R^{2c} and R^{4c} are each an organic group having a long-chain alkyl group having 17 or more carbon atoms, and R^{1c} and R^{3c} are each a hydrogen atom.

n3 is an integer of 4 to 20, and preferably an integer of 4 to 6, and is more preferably 5 or 6.

In a particularly preferable embodiment, R^{3c} and R^{3c} are each —O-R^{12c}, and R^{2c} and R^{4c} are each a hydrogen atom, or R^{2c} and R^{4c} are each —O-R^{12c}, and R^{1c} and R^{3c} is each a hydrogen atom, R^{12c} is a C₁₇₋₃₀ alkyl group, and preferably a C₁₈₋₃₀ alkyl group or a C₂₆₋₃₀ alkyl group, and n3 is an integer of 4 to 6, and is preferably 5 or 6.

The molecular weight of the compound represented by Formula (1C) above can be preferably 500 to 10,000, and more preferably 1,000 to 5,000.

When the compound represented by Formula (1C) above is formed into a film, the static contact angle of the resulting film with water is 80° or more, and preferably 90° or more.

The compound represented by Formula (1C) above can be produced by the same method as for the compound represented by Formula (1A) above. For example, the compound represented by Formula (1C) above can be obtained by introducing a hydroxyl group at a predetermined position of a pillar[n]arene and reacting the hydroxyl group with a tosylate, a halide, or the like of an organic group having a long-chain alkyl group having 17 or more carbon atoms.

In a fourth aspect, the present disclosure provides a compound represented by Formula (1D) below:

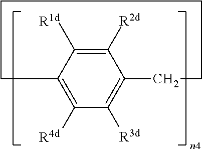

wherein R^{1d} is each independently at each occurrence a hydrogen atom or an organic group, R^{2d} is each independently at each occurrence a hydrogen atom or an organic group, R^{3d} is each independently at each occurrence a hydrogen atom or an organic group, and R^{4d} is each independently at each occurrence a hydrogen atom or an organic group, provided that at least one of R^{1d}, R^{2d}, R3d, and R^{4d} is an organic group having an alkyl group, and the alkyl group has an SP value of 8.2 or more, and n4 is an integer of 4 to 20.

In Formula (1D) above, Rid is each independently at each occurrence a hydrogen atom or an organic group, R2d is each independently at each occurrence a hydrogen atom or an organic group, R^{3d} is each independently at each occurrence a hydrogen atom or an organic group, R^{4d} is each independently at each occurrence a hydrogen atom or an organic group, at least one of R^{1d}, R^{2d}, R^{3d}, and R^{4d} is an organic group having an alkyl group, and the alkyl group has an SP value of 8.2 or more.

The compound represented by Formula (1D) can have high miscibility, heat resistance, and water repellency by having an organic group having an alkyl group having an SP value of 8.2 or more.

The organic group is defined as described for the organic group in Formula (1A) above.

In one embodiment, R^{1d} and R^{3d} are each independently an organic group, and at least one of R^{1d} and R^{3d} is an organic group having an alkyl group having an SP value of 8.2 or more, or R^{2d} and R^{4d} are each independently an organic group, and at least one of R^{2d} and R^{4d} is an organic group having an alkyl group having an SP value of 8.2 or more.

In one embodiment, at least two of R^{1d}, R^{2d}, R^{3d}, and R^{4d} are each an organic group having an alkyl group having an SP value of 8.2 or more.

In one embodiment, two of R^{1d}, R^{2d}, R3d, and R^{4d} are each an organic group having an alkyl group having an SP value of 8.2 or more.

In one embodiment, R^{1d} and R^{3d} are each independently an organic group having an alkyl group having an SP value of 8.2 or more, or R^{2d} and R^{4d} are each independently an organic group having an alkyl group having an SP value of 8.2 or more.

In one embodiment, R^{2d} and R^{4d} are each a hydrogen atom, or R^{1d} and R^{3d} are each a hydrogen atom.

In a preferable embodiment, R^{1d} and R^{3d} are each independently an organic group having an alkyl group having an SP value of 8.2 or more, and R^{2d} and R^{4d} are each a hydrogen atom, or R^{2d} and R^{4d} are each independently an organic group having an alkyl group having an SP value of 8.2 or more, and R^{1d} and R^{3d} are each a hydrogen atom.

In a more preferable embodiment, R^{1d} and R^{3d} are each an organic group having an alkyl group having an SP value of 8.2 or more, and R^{2d} and R^{4d} are each a hydrogen atom, or R^{2d} and R^{4d} are each an organic group having an alkyl group having an SP value of 8.2 or more, and Rid and R^{3d} are each a hydrogen atom.

The SP value of the alkyl group is preferably 8.2 to 8.4.

In a preferable embodiment, the organic group having an alkyl group is preferably a group represented by —O-R^{12d} wherein R^{12d} is a long-chain alkyl group having 17 or more carbon atoms, preferably a C₁₇₋₃₀ alkyl group, and preferably a C₁₈₋₃₀ alkyl group or a C₂₆₋₃₀ alkyl group.

For example, the long-chain alkyl group may be linear or branched, and is preferably linear.

n4 is an integer of 4 to 20, and preferably an integer of 4 to 6, and is more preferably 5 or 6.

In a particularly preferable embodiment, the compound according to any one of claims 30 to 35, wherein R^{1d} and R^{3d} are each —O-R^{12d}, and R^{2d} and R^{4d} are each a hydrogen atom, or R^{2d} and R^{4d} are each —O-R^{12d}, and R^{1d} and R^{3d} is each a hydrogen atom, R^{12d} is a C₁₇₋₃₀ alkyl group, and preferably a C₁₈₋₃₀ alkyl group or a C₂₆₋₃₀ alkyl group, and n4 is an integer of 4 to 6, and is preferably 5 or 6.

The molecular weight of the compound represented by Formula (1D) above can be preferably 500 to 10,000, and more preferably 1,000 to 5,000.

When the compound represented by Formula (1D) above is formed into a film, the static contact angle of the resulting film with water is 80° or more, and preferably 90° or more.

The compound represented by Formula (1D) above can be produced by the same method as for the compound represented by Formula (1A) above. For example, the compound represented by Formula (1D) above can be obtained by introducing a hydroxyl group at a predetermined position of a pillar[n]arene and reacting the hydroxyl group with a tosylate, a halide, or the like of an organic group having an alkyl group.

The present disclosure provides a composition including a compound represented by Formula (1A), (1B), (1C), or (1D) above.

The composition of the present disclosure may include the compounds of the present disclosure singly or may include two or more thereof in combination.

For example, the composition of the present disclosure may be a liquid or a solid.

In the composition of the present disclosure, each component may be included as a so-called inclusion, which is what is obtained by including a further component in the pillararene of the present disclosure, or for example, may be a so-called surface adhered substance, which is what is obtained by adhering a further component to the pillararene of the present disclosure.

The composition of the present disclosure may include a solvent in addition to the compound of the present disclosure.

Examples of the solvent include water; an aliphatic hydrocarbon such as hexane, cyclohexane, heptane, octane, nonane, decane, undecane, dodecane, or a mineral spirit; an aromatic hydrocarbon such as benzene, toluene, xylene, naphthalene, or solvent naphtha; an ester such as methyl acetate, ethyl acetate, propyl acetate, n-butyl acetate, isopropyl acetate, isobutyl acetate, cellosolve acetate, propylene glycol methyl ether acetate, carbitol acetate, diethyl oxalate, ethyl pyruvate, ethyl-2-hydroxybutyrate, ethyl acetoacetate, amyl acetate, methyl lactate, ethyl lactate, methyl 3-methoxypropionate, ethyl 3-methoxypropionate, methyl 2-hydroxyisobutyrate, or ethyl 2-hydroxyisobutyrate; a ketone such as acetone, methyl ethyl ketone, methyl isobutyl ketone, 2-hexanone, cyclohexanone, methylamino ketone, or 2-heptanone; a glycol ether such as ethyl cellosolve, methyl cellosolve, methyl cellosolve acetate, ethyl cellosolve acetate, propylene glycol monomethyl ether, propylene glycol monoethyl ether, propylene glycol monobutyl ether, propylene glycol monomethyl ether acetate, propylene glycol monoethyl ether acetate, propylene glycol monobutyl ether acetate, dipropylene glycol dimethyl ether, or ethylene glycol monoalkyl ether; an alcohol such as methanol, ethanol, iso-propanol, n-butanol, isobutanol, tert-butanol, sec-butanol, 3-pentanol, octyl alcohol, 3-methyl-3-methoxybutanol, or tert-amyl alcohol; a glycol such as ethylene glycol or propylene glycol; a cyclic ether such as tetrahydrofuran, tetrahydropyran, or dioxane; an amide such as N,N-dimethylformamide or N,N-dimethylacetamide; an ether alcohol such as methyl cellosolve, cellosolve, isopropyl cellosolve, butyl cellosolve, or diethylene glycol monomethyl ether; diethylene glycol monoethyl ether acetate; and a fluorine-containing solvent such as 1,1,2-trichloro-1,2,2-trifluoroethane, 1,2-dichloro-1,1,2,2-tetrafluoroethane, dimethylsulfoxide, 1,1-dichloro-1,2,2,3,3-pentafluoropropane (HCFC225), ZEORORA H, HFE7100, HFE7200, HFE7300, CF₃CH₂OH, CF₃CF₂CH₂OH, or (CF₃)₂CHOH. These may be used singly or in combinations of two or more.

When the composition includes a solvent, for example, the compound represented by Formula (1A), (1B), (1C), or (1D) may be a substance swollen by the solvent.

For example, the solvent may be a gas. That is, the composition of the present disclosure may include a mixture of the compound represented by Formula (1A), (1B), (1C), or (1D) above and a gas of the solvent.

The solvent as a gas can be preferably a hydrocarbon, more preferably a C₄₋₁₀ hydrocarbon such as butane, pentane, hexane, cyclohexane, heptane, octane, nonane, or decane, and further preferably pentane or hexane. By including a gas of such a solvent, the contact angle of the film obtained by using the composition of the present disclosure can be improved.

The composition of the present disclosure may include a thermoplastic resin such as a non-fluororesin or a fluororesin.

Examples of the non-fluororesin include a polyolefin-based resin such as polyethylene (for example, linear low-density polyethylene, low-density polyethylene, high-density polyethylene, or ultra-high molecular weight polyethylene), polypropylene including various structural isomers (syndiotactic, isotactic, and atactic structures), poly-(4-methylpentene-1), ethylene-propylene copolymer, ethylene-vinyl acetate copolymer (EVA), a chlorinated polyethylene-based resin, or a modified polyolefin, a cycloolefin resin, a polyvinyl chloride-based resin such as polyvinyl chloride, polyvinylidene chloride, a polystyrene or polystyrene derivative-based resin including various structural isomers, polyamide, polyimide, polyamideimide, polycarbonate, an ionomer, an acrylic resin such as polymethyl methacrylate (PMMA) including various structural isomers, acrylic-styrene copolymer (AS resin), acrylonitrile-butadiene-styrene copolymer (ABS resin), butadiene-styrene copolymer, ethylene-vinyl alcohol copolymer (EVOH), a polyester such as polyethylene terephthalate (PET), polybutylene terephthalate (PBT), or polycyclohexane terephthalate (PCT), polyphenylene sulfide (PPS), polyether sulfone, polyether, polyether ketone (PEK), polyether ether ketone (PEEK), polyether ketone ketone (PEKK) polyetherimide, polyacetal (POM), polyphenylene oxide, modified polyphenylene oxide, polyarylate, aromatic polyester (liquid crystal polymer), a polyurethane-based resin, epoxy resin, phenolic resin, urea resin, melamine resin, unsaturated polyester, silicone resin, polydimethyl silicone (PDMS), polyurethane, a biodegradable resin such as polylactic acid (PLA) or polycaprolactone, or a copolymer, a mixture, a polymer alloy, or the like including the above. Examples of the fluororesin include polytetrafluoroethylene (PTFE), tetrafluoroethylene (TFE)/perfluoroalkyl vinyl ether (PAVE) copolymer [PFA], TFE/hexafluoropropylene (HFP) copolymer [FEP], ethylene [Et]/TFE copolymer [ETFE], Et/TFE/HFP copolymer, polychlorotrifluoroethylene [PCTFE], chlorotrifluoroethylene (CTFE)/TFE copolymer, Et/CTFE copolymer, polyvinylidene fluoride [PVDF], vinylidene fluoride (VDF)/TFE copolymer, and polyvinyl fluoride [PVF].

The composition of the present disclosure may include a filler.

Examples of the filler include, in addition to silica and carbon black, a metal oxide such as calcium oxide, titanium oxide, aluminum oxide, or magnesium oxide; a metal hydroxide such as magnesium hydroxide, aluminum hydroxide, or calcium hydroxide; a carbonate such as magnesium carbonate, aluminum carbonate, calcium carbonate, or barium carbonate; a silicate such as magnesium silicate, calcium silicate, sodium silicate, or aluminum silicate; a sulfate such as aluminum sulfate, calcium sulfate, or barium sulfate; synthetic hydrotalcite; a metal sulfide such as molybdenum disulfide, iron sulfide, or copper sulfide; diatomaceous earth, asbestos, lithopone (zinc sulfide/barium sulfide), graphite, carbon fluoride, calcium fluoride, coke, a fine quartz powder, talc, a mica powder, wollastonite, a carbon fiber, an aramid fiber, various whiskers, a glass fiber, an organic reinforcing agent, an organic filler, polytetrafluoroethylene, mica, celite, and clay. These may be used singly or as a mixture of two or more.

The compounds and the composition of the present disclosure can be used in a wide range of applications, such as a compatibilizer, an adhesive, a surface-treating agent (for example, water repellent), a highly heat-resistant material, an adsorptive separation material, and a material for a formed article.

The compounds and the composition of the present disclosure can each be formed into a formed article. Therefore, the disclosure also provides a formed article formed from any of the compounds and the composition of the disclosure.

The method for forming the compounds and the composition of the present disclosure is not limited, and may be a general forming method such as extrusion forming, injection molding, press forming, vacuum forming, transfer molding, casting, or the like.

The shape of the resulting formed article is not limited, and for example, may be any desired shape such as block-like, sheet-like, film-like, rod-like, or various other shapes depending on the applications.

For example, when obtaining a film, the pillararene of the present disclosure can be dissolved in a good solvent, cast on a substrate, and heated to the boiling point of the solvent or higher to volatilize the solvent to form a film.

A formed article of any of the compounds or the composition of the present disclosure has high heat resistance and water repellency.

A film obtained by casting any of the compounds or the composition of the present disclosure can be treated with a gas of a hydrocarbon, preferably a C₄₋₁₀ hydrocarbon such as butane, pentane, hexane, cyclohexane, heptane, octane, nonane, or decane, more preferably pentane or hexane to improve the water contact angle.

When using any of the compounds or the composition of the present disclosure as a surface-treating agent, various substrates can be treated. Therefore, the disclosure provides a material including a substrate treated with any of the compounds and/or the composition of the disclosure.

A substrate that can be treated with any of the compounds and the composition of the present disclosure can be composed of, for example, glass, a resin (which, for example, may be a natural or synthetic resin, such as a common plastic material), a metal, a ceramic, a semiconductor (silicon, germanium, or the like), a fiber (woven fabric, nonwoven fabric, or the like), fur, leather, a wood, a ceramic material, a stone material, a building member, a sanitary material, an adsorptive separation material, or any suitable material.

A substrate treated with any of the compounds or the composition of the present disclosure has high heat resistance and water repellency.

The present disclosure provides a liquid-repellent composition including a pillararene represented by Formula (1E) below and a hydrocarbon compound:

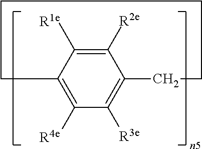

wherein R^{1e}, R^{2e}, R^{3e}, and R^{4e} are each independently a hydrogen atom or an organic group, and n5 is 5 or 6.

The formed article of the liquid-repellent composition has high water repellency.

The organic group is an organic group having preferably 1 to 30 carbon atoms, and more preferably 1 to 20 carbon atoms.

The liquid-repellent composition may further include a solvent.

Examples of the solvent include the same solvents as described for the composition including the compound represented by Formula (1A), (1B), (1C), or (1D) above. The solvent included in the liquid-repellent composition can be a C₄₋₁₀ hydrocarbon such as butane, pentane, hexane, cyclohexane, heptane, octane, nonane, or decane, and further preferably pentane or hexane.

The hydrocarbon compound included in the liquid-repellent composition can be preferably a C₄₋₁₀ hydrocarbon such as butane, pentane, hexane, cyclohexane, heptane, octane, nonane, or decane, and further preferably pentane or hexane.

The hydrocarbon compound included in the liquid-repellent composition may be the same as or different from the solvent.

In the liquid-repellent composition, the pillararene represented by Formula (1E) and the hydrocarbon compound may be included as a so-called inclusion, which is what is obtained by including the hydrocarbon compound in the pillararene represented by Formula (1E), or for example, may be a so-called surface adhered substance, which is what is obtained by adhering the hydrocarbon compound to the pillararene.

When the liquid-repellent composition includes a solvent, for example, the pillararene represented by Formula (1E) may be a substance swollen by the solvent.

The inclusion and the surface adhered substance can be obtained by treating the pillararene represented by Formula (1E) with a gas of a hydrocarbon compound.

The compounds of the present disclosure have been described in detail above. The methods for producing the compounds of the present disclosure, and the like are not limited to those given as examples above.

The present disclosure includes the following aspects. [1] A compound represented by Formula (1A) below:

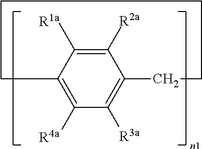

wherein R^{1a} is each independently at each occurrence a hydrogen atom or an organic group, R^{2a} is each independently at each occurrence a hydrogen atom or an organic group, R^{3a} is each independently at each occurrence a hydrogen atom or an organic group, R^{4a} is each independently at each occurrence a hydrogen atom or an organic group, provided that at least one of R^{1a}, R^{2a}, R^{3a}, and R^{4a} is an organic group having 2 or more carbon atoms and having a fluoroalkyl group or a fluorophenyl group, and n1 is an integer of 4 to 20. [2] The compound according to [1] above, wherein R^{1a} and R^{3a} are each independently an organic group, provided that at least one of R^{1a} and R^{3a} is an organic group having 2 or more carbon atoms and having a fluoroalkyl group or a fluorophenyl group, or R^{2a} and R^{4a} are each independently an organic group, provided that at least one of R^{2a} and R^{4a} is an organic group having 2 or more carbon atoms and having a fluoroalkyl group or a fluorophenyl group. [3] The compound according to [1] or [2] above, wherein R^{1a} and R^{3a} are each independently an organic group having 2 or more carbon atoms and having a fluoroalkyl group or a fluorophenyl group, or R^{2a} and R^{4a} are each independently an organic group having 2 or more carbon atoms and having a fluoroalkyl group or a fluorophenyl group. [4] The compound according to any one of [1] to [3] above, wherein R^{2a} and R^{4a} are each a hydrogen atom, or R^{1a} and R^{3a} are each a hydrogen atom. [5] The compound according to any one of [1] to [4] above, wherein n1 is an integer of 4 to 6. [6] The compound according to any one of [1] to [5] above, the organic group having 2 or more carbon atoms and having a fluoroalkyl group is —(Oₚ₁-R^{11a}_{q1})-Rf^{a}, R^{11a} is each independently at each occurrence a C₁₋₁₀ alkylene group or —CONH—, Rf^{a} is a C₁₋₁₀ fluoroalkyl group, p1 is an integer of 0 to 2, q1 is an integer of 0 to 3, and in (Oₚ₁-R^{11a}_{q1}), occurrence order of O and R^{11a} is not limited. [7] The compound according to any one of [1] to [6] above, wherein the organic group having 2 or more carbon atoms and having a fluoroalkyl group is —O-R^{11a}-(O-R^{11a})ᵣᵣ-Rf^{a}, R^{11a} is each independently a C₁₋₁₀ alkylene group or —CONH—, Rf^{a} is a C₁₋₁₀ fluoroalkyl group, and r1 is 0 or 1. [8] The compound according to any one of [1] to [7] above, wherein the fluoroalkyl group is a perfluoroalkyl group. [9] The compound according to any one of [1] to [8] above, wherein R^{1a} and R^{3a} are each —O-R^{11a}-(O-R^{11a})ᵣ₁-Rf^{a}, and R^{2a} and R^{4a} are each a hydrogen atom, or

R^{2a} and R^{4a} are each —O-R^{11a}-(O-R^{11a})ᵣ₁-Rf^{a}, and R^{1a} and R^{3a} are each a hydrogen atom, R^{11a} is each independently a C₁₋₁₀ alkylene group or —CONH—, Rf^{a} is a C₁₋₁₀ perfluoroalkyl group, r1 is 0 or 1, and n1 is an integer of 4 to 6. [10] The compound according to any one of [1] to [5] above, the organic group having 2 or more carbon atoms and having a fluorophenyl group is a group represented by the following formula: —(Oₚ₃-R^{11p}_{q3})-Rf^{p} wherein R^{11p} is each independently at each occurrence a C₁₋₁₀ alkylene group, Rf^{p} is a phenyl group substituted by 1 to 5 fluorine, p3 is an integer of 0 to 2, q3 is an integer of 0 to 3, and in (Oₚ₃-R^{11p}_{q3}), occurrence order of O and R^{11p} is not limited. [11] The compound according to any one of [1] to [5] above, wherein the organic group having 2 or more carbon atoms and having a fluorophenyl group is a group represented by the following formula: —O-R^{11p}-Rf^{p} wherein R^{11p} is each independently at each occurrence a C₁₋₁₀ alkylene group, and Rf^{p} is a phenyl group substituted with 1 to 5 fluorine atoms. [12] The compound according to any one of [1] to [5] above, wherein R^{1a} and R^{3a} are each —O-R^{11p}-Rf^{p}, and R^{2a} and R^{4a} are each a hydrogen atom, or R^{2a} and R^{4a} are —O-R^{11p}-Rf^{p}, and R^{1a} and R^{3a} are each a hydrogen atom, R^{11p} is each independently a C₁₋₆ alkylene group, Rf^{p} is a phenyl group substituted with 1 to 5 fluorine atoms, and n1 is an integer of 4 to 6. [13] The compound according to any one of [1] to [12] above, wherein when the compound is formed into a film, a static contact angle of the film with water is 80° or more. [14] A compound represented by Formula (1B) below:

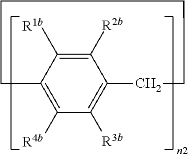

wherein R^{1b} is each independently at each occurrence a hydrogen atom or an organic group, R^{2b} is each independently at each occurrence a hydrogen atom or an organic group, R^{3b} is each independently at each occurrence a hydrogen atom or an organic group, and R^{4b} is each independently at each occurrence a hydrogen atom or an organic group, provided that at least one of R^{1b}, R^{2b}, R^{3b}, and R^{4b} is an organic group having a fluorine atom, and the organic group having a fluorine atom has an SP value of 8.0 or less, and n2 is an integer of 4 to 20. [15] The compound according to [14] above, wherein R^{1b} and R^{3b} are each independently an organic group, provided that at least one of Rib and R^{3b} is an organic group having a fluorine atom and having an SP value of 8.0 or less, or R^{2b} and R^{4b} are each independently an organic group, provided that at least one of R^{2b} and R^{4b} is an organic group having a fluorine atom and having an SP value of 8.0 or less. [16] The compound according to [14] or [15] above, wherein R^{1b} and R^{3b} are each independently an organic group having a fluorine atom and having an SP value of 8.0 or less, or R^{2b} and R^{4b} are each independently an organic group having a fluorine atom and having an SP value of 8.0 or less. [17] The compound according to any one of [14] to [16] above, wherein R^{2b} and R^{4b} are each a hydrogen atom, or R^{1b} and R^{3b} are each a hydrogen atom. [18] The compound according to any one of [14] to [17] above, wherein n2 is an integer of 4 to 6. [19] The compound according to any one of [14]to [18] above, wherein the SP value of the organic group having a fluorine atom is 4.0 to 7.5. [20] The compound according to any one of [14] to [19] above, wherein the organic group having a fluorine atom has a C₁₋₁₀ fluoroalkyl group. [21] The compound according to [30] above, wherein the C₁₋₁₀ fluoroalkyl group is a C₁₋₁₀ perfluoroalkyl group. [22] The compound according to any one of [14] to [21] above, wherein R^{1b} and R^{3b} are each —O-R^{1b}-(O-R^{11b})ᵣ₂-Rf^{b}, and R^{2b} and R^{4b} are each a hydrogen atom, or R^{2b} and R^{4b} are each —O-R^{11b}- (O-R^{11b})ᵣ₂-Rf^{b}, and R^{1b} and R^{3b} are each a hydrogen atom, R^{11b} is each independently a C₁₋₁₀ alkylene group, Rf^{b} is a C₁₋₁₀ perfluoroalkyl group, r2 is 0 or 1, and n2 is an integer of 4 to 6. [23] The compound according to any one of [14] to [22] above, wherein when the compound is formed into a film, a static contact angle of the film with water is 80° or more. [24] A compound represented by Formula (1C) below:

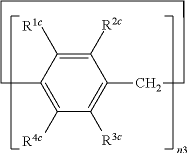

wherein R^{1c} is each independently at each occurrence a hydrogen atom or an organic group, R^{2c} is each independently at each occurrence a hydrogen atom or an organic group, R^{3c} is each independently at each occurrence a hydrogen atom or an organic group, R^{4c} is each independently at each occurrence a hydrogen atom or an organic group, provided that at least one of R^{1c}, R^{2c}, R^{3c}, and R^{4c} is an organic group having a long-chain alkyl group having 17 or more carbon atoms, and n3 is an integer of 4 to 20. [25] The compound according to [24] above, wherein R^{1c} and R^{3c} are each independently an organic group, provided that at least one of R^{1c} and R^{3c} is an organic group having a long-chain alkyl group having 17 or more carbon atoms, or R^{2c} and R^{4c} are each independently an organic group, provided that at least one of R^{2c} and R^{4c} is an organic group having a long-chain alkyl group having 17 or more carbon atoms. [26] The compound according to or above, wherein R^{1c} and R^{3c} are each independently an organic group having a long-chain alkyl group having 17 or more carbon atoms, and R^{2c} and R^{4c} are each independently an organic group having a long-chain alkyl group having 17 or more carbon atoms. [27] The compound according to any one of [24] to [26] above, wherein the number of carbon atoms in the long-chain alkyl group is 17 to 30. [28] The compound according to any one of [24] to [27] above, wherein R^{1c} and R^{3c} are each independently —O-R^{12c} , or R^{2c} and R^{4c} are each independently —O-R^{12c} , and R^{12c} is a C₁₇₋₃₀ alkyl group. [29] The compound according to any one of [24] to [28] above, wherein R^{2c} and R^{4c} are each a hydrogen atom, or R^{1c} and R^{3c} are each a hydrogen atom. [30] The compound according to any one of [24] to [29] above, wherein n3 is an integer of 4 to 6. [31] The compound according to any one of [24] to [30] above, wherein R^{1c} and R^{3c} are each —O-R^{12c}, and R^{2c} and R^{4c} are each a hydrogen atom, or R^{2c} and R^{4c} are each —O-R^{12c}, and R^{1c} and R^{3c} is each a hydrogen atom, R^{12c} is a C₁₇₋₃₀ alkyl group, and n3 is an integer of 4 to 6. [32] The compound according to any one of [24] to [31] above, wherein when the compound is formed into a film, a static contact angle of the film with water is 80° or more. [33] A compound represented by Formula (1D) below:

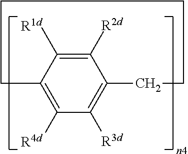

wherein R^{1d} is each independently at each occurrence a hydrogen atom or an organic group, R^{2d} is each independently at each occurrence a hydrogen atom or an organic group, R^{3d} is each independently at each occurrence a hydrogen atom or an organic group, and R^{4d} is each independently at each occurrence a hydrogen atom or an organic group, provided that at least one of R^{1d}, R^{2d}, R3d, and R^{4d} is an organic group having an alkyl group, and the alkyl group has an SP value of 8.2 or more, and n4 is an integer of 4 to 20. [34] The compound according to [33] above, wherein R^{1d} and R^{3d} are each independently an organic group, provided that at least one of R^{1d} and R^{3d} is an organic group having an alkyl group having an SP value of 8.2 or more, or R^{2d} and R^{4d} are each independently an organic group, provided that at least one of R^{2d} and R^{4d} is an organic group having an alkyl group having an SP value of 8.2 or more. [35] The compound according to or above, wherein R^{1d} and R^{3d} are each independently an organic group having an alkyl group having an SP value of 8.2 or more, or R^{2d} and R^{4d} are each independently an organic group having an alkyl group having an SP value of 8.2 or more. [36] The compound according to any one of [33] to [35] above, wherein R^{2d} and R^{4d} are each a hydrogen atom, or R^{1d} and R^{3d} are each a hydrogen atom. [37] The compound according to any one of [33] to [36] above, wherein n4 is an integer of 4 to 6. [38] The compound according to any one of [33] to [37] above, wherein the SP value of the alkyl group is 8.2 to 8.4. [39] The compound according to any one of [33] to [38] above, wherein R^{1d} and R^{3d} are each —O-R^{12d}, and R^{2d} and R^{4d} are each a hydrogen atom, or R^{2d} and R^{4d} are each —O-R^{12d} , and R^{1d} and R^{3d} is each a hydrogen atom, R^{12d} s a C₁₇₋₃₀ alkyl group, and n4 is an integer of 4 to 6. [40] The compound according to any one of [33] to [39] above, wherein when the compound is formed into a film, a static contact angle of the film with water is 80° or more. [41] A composition comprising at least one of the compound according to any one of [1] to [40] above. [42] A formed article of the compound according to any one of [1] to [40] above. [43] A formed article formed from the composition according to [41] above. [44] The formed article according to [42] or [43] above, wherein the formed article is a film. [45] A material comprising a substrate treated with the compound according to any one of [1] to [40] above or the composition according to above. [46] A liquid-repellent composition comprising a pillararene represented by Formula (1E) below and a hydrocarbon compound:

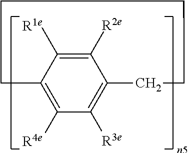

where R^{1e}, R^{2e}, R^{3e}, and R^{4e} are each independently a hydrogen atom or an organic group, and n5 is 5 or 6. [47] The liquid-repellent composition according to [46] above, wherein the organic group is an organic group having 1 to 30 carbon atoms. [48] The liquid-repellent composition of [46] or [47] above, wherein the liquid-repellent composition further comprises a solvent.

### EXAMPLES

The compounds of the present disclosure will be more specifically described through the following Examples, but the present disclosure is not limited to these Examples.

Synthesis Example 1

Dehydrated dimethylformamide (30 mL) and dehydrated tetrahydrofuran (30 mL) were added to pillar[5]arene in a hydroquinone form (1.30 g, 2.13 mmol), and then NaH (1.33 g, 66.7 mmol) was added. 1-Tosyloxy-4,4,4-trifluorobutane (12.1 g, 42.9 mmol) was added, and then the reaction was carried out at 60° C. for 72 hours. The temperature was returned to room temperature, and then methanol was added to quench the reaction. After concentration, separation was carried out with dichloromethane and saturated brine, and Na₂SO₄ was added to the chloroform layer for drying. Na₂SO₄ was removed by filtration, and concentration was carried out. Purification by silica column chromatography (hexane:dichloromethane=2:1) gave the following substituted pillar[5]arene (yield of 15%).

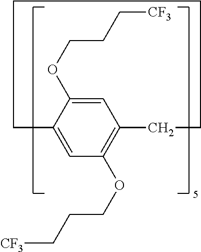

### CF₃[5]: 1H NMR (400 MHz, CDCl3) δ 6.75 (s, 10H), 3.89 (t, J=10.8 Hz, 20H), 3.74 (s, 10H), 2.29-2.41 (m, 20H), 2.01-2.08 (m, 20H)

Synthesis Example 2

The following substituted pillar[5]arene (yield of 13%) was obtained in the same manner as in Synthesis Example 1 except that 4,4,5,5,5-pentafluoropentyl-p-toluenesulfonate (14.3 g, 42.9 mmol) was used instead of 1-tosyloxy-4,4,4-trifluorobutane.

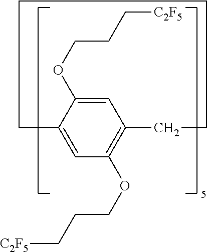

C₂F₅[5]: 1H NMR (400 MHz, CDCl3) δ 6.76 (s, 10H), 3.75-4.03 (m, 20H), 3.73 (s, 10H), 2.25-2.34 (m, 20H), 2.08-2.11 (m, 20H)

Synthesis Example 3

The following substituted pillar[5]arene (yield of 5.3%) was obtained in the same manner as in Synthesis Example 1 except that 8-[(6-bromohexyl)oxy]-10 1,1,1,2,2,3,3,4,4,5,5,6,6-tridecafluoro-octane (22.6 g, 42.9 mmol) was used instead of 1-tosyloxy-4,4,4-trifluorobutane.

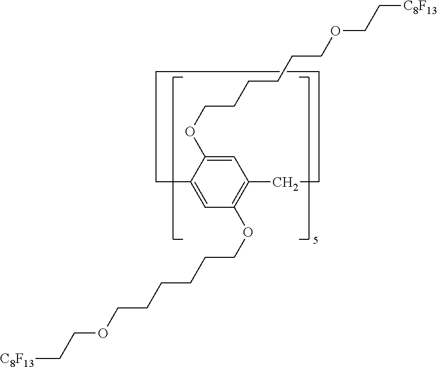

C₆F₁₃[5]: 1H NMR (400 MHz, CDCl3) δ 6.86 (bs, 10H), 3.24-4.21 (m, 89H), 2.38 (bs, 22H), 1.51-2.10 (m, 31H), 1.45 (bs, 28H)

Synthesis Example 4

The following substituted pillar[6]arene (yield of 24%) was obtained in the same manner as in Synthesis Example 1 except that pillar[6]arene in a hydroquinone form (1.30 g, 1.77 mmol), was used instead of pillar[5]arene in a hydroquinone form, and 4,4,5,5,5-pentafluoropentyl-p-toluenesulfonate (14.3 g, 42.9 mmol) was used instead of 1-tosyloxy-4,4,4-trifluorobutane.

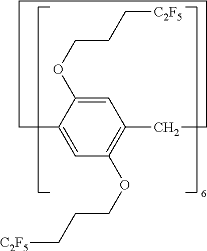

C₂F₅[6]: 1H NMR (400 MHz, CDCl3) δ 6.66 (s, 12H), 3.78-3.82 (m, 36H), 2.14-2.27 (m, 24H), 1.93-2.04 (m, 24H)

Synthesis Example 5

Paraformaldehyde (0.31 g, 10 mmol) was added to a solution of 1,4-dipentyloxybenzene (1.66 g, 10 mmol) in 1,2-dichloroethane (20 mL) in a nitrogen atmosphere. Next, boron trifluoride diethyl etherate (1.25 mL, 10 mmol) was added, and the resulting mixture was stirred at a temperature of 30° C. for 3 hours. The solution was poured into methanol, and the precipitate was collected by filtration. The precipitate was dissolved in trichloromethane, and the undissolved portion was removed by filtration. Trichloromethane was removed under reduced pressure, and the target compound (0.413 g, yield of 23.2%) was crystallized with acetone.

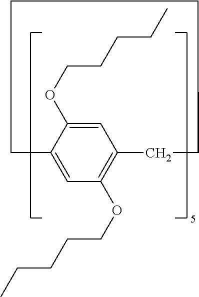

1H NMR (CDCl3, 400 MHz, TMS) δ 6.72 (s, 10H), 3.82 (q, J=7.1 Hz, 20H), 3.76 (s, 10H), 1.25 (t, J=7.1 Hz, 30H) 13C NMR (CDCl3, 100 MHz, TMS) δ 149.8, 128.5, 115.1, 63.8, 15 29.8, 15.0

Synthesis Example 6

Pillar[6]arene in a hydroquinone form (0.582 g, 0.795 mmol), was dissolved in acetonitrile (60 mL). Next, pentafluorobenzyl bromide (5.00 g, 19.2 mmol)) was added, and further, K₂CO₃ (1.65 g, 11.9 mmol) and KI (0.319 g, 1.92 mmol) were added. The resulting mixture was heated to 60° C. and allowed to react for 72 hours. The target product was isolated by silica gel chromatography (dichloromethane:hexane=20 to 25:80 to 75). ¹H NMR and HPLC demonstrated that the target compound was obtained.

1H NMR (CDCl3, 400 MHz, TMS) δ 6.71 (s, 12H), 4.80 (s, 24H), 3.70 (s, 12H)

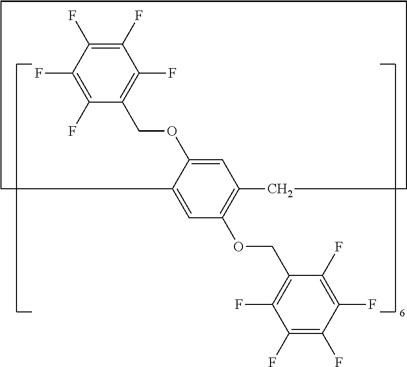

Synthesis Example 7

Pillar[5]arene in a hydroquinone form (0.305 g, 0.5 mmol), was dissolved in dimethylsulfoxide (5 mL). After that, stearyl isocyanate (1.478 g, 5 mmol) was added, and 1 drop of dibutyltin laurate as a catalyst was added. The resulting mixture was heated to 40° C. and allowed to react for 6 hours. The reaction solution and the precipitate were cleaned with hexane and water in presented order to obtain the target compound. FT-IR demonstrated that the target compound was obtained, by disappearance of the peak derived from pillararene in a hydroquinone form, that was broad around 3000 to 3500 cm⁻¹, disappearance of the C═O peak derived from stearyl isocyanate at 2266 cm⁻¹, and generation of the C═O peak derived from the urethane bond around 1708 cm⁻¹.

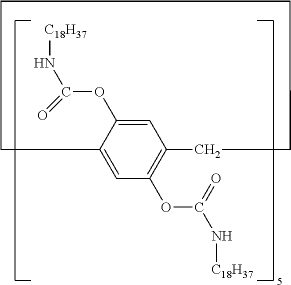

<SP value and miscibility>(SP value)

The SP values of side chains of the pillararenes of

Synthesis Examples 1 to 4 were calculated by the Fedors method described above. Results thereof are shown in the following table.

(Miscibility)

As shown in the following table, the SP values of the side chain structures of Synthesis Examples 1 to 4 were in the range of 6.9 to 7.4. On the other hand, for example, when a fluororesin having an SP value of 5.9 is selected, it is considered that the pillararene compound and the fluororesin have good miscibility because the SP values of the both are close to each other.

TABLE 1

| | Side chain structure | SP value |
|---|---|---|
| Synthesis Example 1 | —O—(CH₂)₃—CF₃ | 7.0 |
| Synthesis Example 2 | —O—(CH₂)₃—C₂F₅ | 6.9 |
| Synthesis Example 3 | —O—(CH₂)₆—O—(CH₂)₂—C₆F₁₃ | 7.4 |
| Synthesis Example 4 | —O—(CH₂)₃—C₂F₅ | 6.9 |

(SP Value)

When the side chain structure is a long-chain alkyloxy group: —O—C₁₇H₃₅, —O—C₂₀H₄₁, —O—C₂₆H₅₃, or —O—C₃₀H₆₁ (all linear), the SP value was calculated by the Fedors method described above. Results thereof are shown in the following table.

(Miscibility)

As shown in the following table, the SP values of the side chain structures of Reference Examples 1 to 4 were 8.4 to 8.5. On the other hand, for example, when a fluororesin having an SP value of 8.1 is selected, it is considered that the pillararene compound and the fluororesin have good miscibility because the SP values of both are close to each other.

TABLE 2

| | Side chain structure | SP value |
|---|---|---|
| Reference Example 1 | —O—C₁₇H₃₅ | 8.4 |
| Reference Example 2 | —O—C₂₀H₄₁ | 8.4 |
| Reference Example 3 | —O—C₂₆H₅₃ | 8.4 |
| Reference Example 4 | —O—C₃₀H₆₁ | 8.5 |

<Film Formation>(Formation of cast film)

The pillararenes obtained in Synthesis Examples 1, 2, 4, and 5 above were dissolved in dichloromethane. The obtained solutions were cast on glass substrates, respectively, dichloromethane was volatilized at room temperature, and then the resulting solids were heated to 160° C. and melted. Next, the resulting melts were cooled at room temperature to obtain cast films of the pillararenes.

(Post-treatment)

A sample bottle containing a solvent and the cast films obtained above were added, the cast films were exposed to solvent vapor for 17 hours, and then air-dried in a fume hood for 2 hours to obtain solvent-treated films. The combinations of cast films and solvents are as follows.

Cast film of Synthesis Example 2: pentane, hexane, heptane, octane

Cast film of Synthesis Example 4: cyclohexane

Cast film of Synthesis Example 5: pentane, hexane

Each of the air-dried samples was dissolved in deuterated chloroform, and the integrated values of the ¹H NMR peaks of the pillararene and the alkane moiety demonstrated that the solvent(s) was(were) included in the pillararene.

(Measurement of Contact Angle)

The contact angles of the cast films obtained from the compounds of Synthesis Examples 1, 2, 4, and 5 and the solvent-treated films obtained above were measured before and after the exposure. The contact angle was measured by using water with a fully automatic compact contact angle meter (P200A-HSM, Meiwafosis Co., Ltd.). Results thereof are shown in the following table.

TABLE 3

| | Initial | | | | | |
|---|---|---|---|---|---|---|
| | contact | | | | | |
| | angle | Contact angle after solvent vapor treatment (°) | | | | |
| | (°) | Pentane | Hexane | Cyclohexane | Heptane | Octane |
| Synthesis | 99 | — | — | — | — | — |
| Example 1 | | | | | | |
| Synthesis | 101 | 121 | 113 | — | 101 | 102 |
| Example 2 | | | | | | |
| Synthesis | 108 | — | — | 110 | — | — |
| Example 4 | | | | | | |
| Synthesis | 97 | 102 | 106 | — | — | — |
| Example 5 | | | | | | |

The above results demonstrated that the films of the pillararenes of Synthesis Examples 1, 2, 4, and 5 had good water repellency. In particular, these demonstrated that the films had much better water repellency when treated with solvent vapor.

<Melting Point>

The melting points of the cast films obtained from the compounds of Synthesis Examples 1, 2, 4, and 5 were measured with DSC7020 (Hitachi High-Tech Science Co., Tokyo, Japan). Results thereof are shown in the following table.

TABLE 4

| | Melting point (°) |
|---|---|
| Synthesis Example 1 | 148 |
| Synthesis Example 2 | 117 |
| Synthesis Example 4 | 87 |
| Synthesis Example 5 | 83 |

The above results demonstrated that the pillararenes of Synthesis Examples 1, 2, and 4, particularly Synthesis Examples 1 and 2, had a high melting point and excellent heat resistance. When any of the compounds is used as an additive to a resin, the high melting point of the compound can suppress softening of the resin. Therefore, the pillararenes of the present disclosure can be used in a wide range of applications such as an application requiring heat resistance.

### INDUSTRIAL APPLICABILITY

The compounds of the present disclosure can be used in a wide range of applications, such as a compatibilizer, an adhesive, a surface-treating agent, a highly heat-resistant material, an adsorptive separation material, and a material for a formed article.

1. A compound represented by Formula (1A) below: 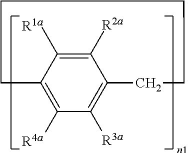
wherein R^{1a} is each independently at each occurrence a hydrogen atom or an organic group, R^{2a} is each independently at each occurrence a hydrogen atom or an organic group, R^{3a} is each independently at each occurrence a hydrogen atom or an organic group, R^{4a} is each independently at each occurrence a hydrogen atom or an organic group, the organic group is —(Oₚ₁R^{11a}_{q1})-Rf^{a} wherein: R^{11a} is each independently at each occurrence a C₁₋₁₀ alkylene group or —CONH—, Rf^{a} is a C₁₋₁₀ fluoroalkyl group, p1 is an integer of 0 to 2, q1 is an integer of 0 to 3, and in (Oₚ₁-R^{11a}_{q1}), occurrence order of O and R^{11a} is not limited, or —(Oₚ₃-R^{11p}_{q3})-Rf^{p}wherein: R^{11p} is each independently at each occurrence a C₁₋₁₀ alkylene group, Rf^{p} is a phenyl group substituted by 1 to 5 fluorine, p3 is an integer of 0 to 2, q3 is an integer of 0 to 3, and in (Oₚ₃-R^{11p}_{q3}), occurrence order of O and R^{11p} is not limited, provided that at least one of R^{1a}, R^{2a}, R^{3a}, and R^{4a} is —(Oₚ₁-R^{11a}_{q1})-Rf^{a} or —(Oₚ₃-R^{11p}_{q3})-Rf^{p}, and n1 is an integer of 4 to 20.

2. The compound according to claim 1, wherein R^{1a} and R^{3a} are each independently —(Oₚ₁-R^{11a}_{q1})-Rf^{a} or —(Oₚ₃-R^{11p}_{q3})-Rf^{p}, or R^{2a} and R^{4a} are each independently —(Oₚ₁-R^{11a}_{q1})-Rf^{a} or —(Oₚ₃-R^{11p}_{q3})-Rf^{p}.

3. The compound according to claim 1, wherein n1 is an integer of 4 to 6.

4. The compound according to claim 1, wherein the organic group is —(Oₚ₁-R^{11a}_{q1})-Rf^{a}, R^{11a} is each independently at each occurrence a C₁₋₁₀ alkylene group or —CONH—, Rf^{a} is a C₁₋₁₀ fluoroalkyl group, p1 is an integer of 0 to 2, q1 is an integer of 0 to 3, and in (Oₚ₁-R^{11a}_{q1}), occurrence order of O and R^{11a} is not limited.

5. The compound according to claim 1, wherein the organic group is —O-R^{11a}—O-R^{11a})ᵣ₁-Rf^{a}, R^{11a} is each independently a C₁₋₁₀ alkylene group or —CONH—, Rf^{a} is a C₁₋₁₀ fluoroalkyl group, and r1 is 0 or 1.

6. The compound according to claim 1, wherein Rf^{a} is a perfluoroalkyl group.

7. The compound according to claim 1, wherein R^{1a} and R^{3a} are each —O-R^{11a}-(O-R^{11a})ᵣ₁-Rf^{a}, and R^{2a} and R^{4a} are each a hydrogen atom, or R^{2a} and R^{4a} are each —O-R^{11a}—(O-R^{11a})ᵣ₁-Rf^{a}, and R^{1a} and R^{3a} are each a hydrogen atom, R^{11a} is each independently a C₁₋₁₀ alkylene group or —CONH—, Rf^{a} is a C₁₋₁₀ perfluoroalkyl group, r1 is 0 or 1, and n1 is an integer of 4 to 6.

8. The compound according to claim 1, wherein the organic group is a group represented by the following formula: —(Oₚ₃-R^{11p}_{q3})-Rf^{p}wherein R^{11p} is each independently at each occurrence a C₁₋₁₀ alkylene group, Rf^{p} is a phenyl group substituted by 1 to 5 fluorine, p3 is an integer of 0 to 2, q3 is an integer of 0 to 3, and in (Oₚ₃-R^{11p}_{q3}), occurrence order of O and R^{11p} is not limited.

9. The compound according to claim 1, wherein the organic group is a group represented by the following formula: —O-R^{11p}-Rf^{p} wherein R^{11p} is each independently at each occurrence a C₁₋₁₀ alkylene group, and Rf^{p} is a phenyl group substituted with 1 to 5 fluorine atoms.

10. The compound according to claim 1, wherein R^{1a} and R^{3a} are each —O-R^{11p}-Rf^{p}, and R^{2a} and R^{4a} are each a hydrogen atom, or R^{2a} and R^{4a} a are —O-R^{11p}-Rf^{p}, and R^{1a} and R^{3a} are each a hydrogen atom, R^{11p} is each independently a C₁₋₆ alkylene group, Rf^{p} is a phenyl group substituted with 1 to 5 fluorine atoms, and n1 is an integer of 4 to 6.

11. The compound according to claim 1, wherein when the compound is formed into a film, a static contact angle of the film with water is 80° or more.

12. A compound represented by Formula (1B) below: 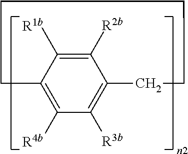
wherein R^{1b} is each independently at each occurrence a hydrogen atom or an organic group, R^{2b} is each independently at each occurrence a hydrogen atom or an organic group, R^{3b} is each independently at each occurrence a hydrogen atom or an organic group, and R^{4b} is each independently at each occurrence a hydrogen atom or an organic group, the organic group is —Oₚ₂-R^{11b}_{q2})-Rf^{b} wherein: R^{11b} is each independently at each occurrence a C₁₋₁₀ alkylene group, Rf^{b} is a C₁₋₁₀ fluoroalkyl group, p2 is an integer of 0 to 2, q2 is an integer of 0 to 3, and in (Oₚ₂-R^{11b}_{q2}), occurrence order of O and R^{11b} is not limited, or —(Oₚ₁-R^{11a}_{q1})-Rf^{p} wherein: R^{11a} is each independently at each occurrence a C₁₋₁₀ alkylene group, Rf^{p} is a phenyl group substituted by 1 to 5 fluorine, p1 is an integer of 0 to 2, q1 is an integer of 0 to 3, and in (Oₚ₁-R^{11a}_{q1}), occurrence order of O and R^{11a} is not limited, provided that at least one of R^{1b}, R^{2b}, R^{3b}and R^{4b} is —(Oₚ₂-R^{11b}_{q2})-Rf^{b} or —(Oₚ₁-R^{11a}_{q1})-Rf^{p}, and —(Oₚ₂-R^{11b}_{q2})-Rf^{b} or - (Oₚ₁-R^{11a}_{q1})-Rf^{p} has an SP value of 8.0 or less, and n2 is an integer of 4 to 20.

13. The compound according to claim 12, wherein R^{1b} and R^{3b} are each independently —(Oₚ₂-R^{11b}_{q2})-Rf^{b} or —(Oₚ₁-R^{11a}_{q1})-Rf^{p}, provided that at least one of R^{1b} and R^{3b} is —(Oₚ₂-R^{11b}_{q2})-Rf^{b} or —(Oₚ₁-R^{11a}_{q1})-Rf^{p} having an SP value of 8.0 or less, or R^{2b} and R^{4b} are each independently —(Oₚ₂-R^{11b}_{q2})-Rf^{b} or —(Oₚ₁-R^{11a}_{q1})-Rf^{p}, provided that at least one of R^{2b} and R^{4b} is —(Oₚ₂-R^{11b}_{q2})-Rf^{b} or —(Oₚ₁-R^{11a}_{q1})-Rf^{p} having an SP value of 8.0 or less.

14. The compound according to claim 12, wherein R^{1b} and R^{3b} are each independently —(Oₚ₂-Rhu 11b_{q2})-Rf^{b} or —(Oₚ₁-R^{11a}_{q1})-Rf^{p} having an SP value of 8.0 or less, or R^{2b} and R^{4b} are each independently —(Oₚ₂-R^{11b}_{q2})-Rf^{b} or —(Oₚ₁-R^{11a}_{q1})-Rf^{p} having an SP value of 8.0 or less.

15. The compound according to claim 12, wherein R^{2b} and R^{4b} are each a hydrogen atom, or R^{1b} and R^{3b} are each a hydrogen atom.

16. The compound according to claim 12, wherein n2 is an integer of 4 to 6.

17. The compound according to claim 12, wherein the SP value of —(Oₚ₂-R^{11b}_{q2})-Rf^{b} or —(Oₚ₁-R^{11a}_{q1})-Rf^{p} is 4.0 to 7.5.

18. The compound according to claim 12, wherein the organic group is: —(Oₚ₂-R^{11b}_{q2})-Rf^{b} wherein: R^{11b} is each independently at each occurrence a C₁₋₁₀ alkylene group, Rf^{b} is a C₁₋₁₀ fluoroalkyl group, p2 is an integer of 0 to 2, q2 is an integer of 0 to 3, and in (Oₚ₂-R^{11b}_{q2}), occurrence order of O and R^{11b} is not limited.

19. The compound according to claim 18, wherein Rf^{b} is a C₁₋₁₀ perfluoroalkyl group.

20. The compound according to claim 12, wherein R^{1b} and R^{3b} are each —O-R^{11b}-(O-R^{11b})ᵣ₂-Rf^{b}, and R^{2b} and R^{4b} are each a hydrogen atom, or R^{2b} and R^{4b} are each —O-R^{11b}-(O-R^{11b})ᵣ₂-Rf^{b}, and R^{1b} and R^{3b} are each a hydrogen atom, R^{11b} is each independently a C₁₋₁₀ alkylene group, Rf^{b} is a C₁₋₁₀ perfluoroalkyl group, r2 is 0 or 1, and n2 is an integer of 4 to 6.

21. The compound according to claim 12, wherein when the compound is formed into a film, a static contact angle of the film with water is 80° or more.

22. A compound represented by Formula (1C) below: 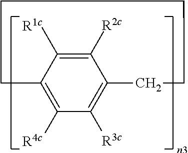
wherein R^{1c} is each independently at each occurrence a hydrogen atom or an organic group, R^{2c} is each independently at each occurrence a hydrogen atom or an organic group, R^{3c} is each independently at each occurrence a hydrogen atom or an organic group, R^{4c} is each independently at each occurrence a hydrogen atom or an organic group, the organic group is each independently —O-R^{12c}, R^{12c} is C₁₇₋₃₀ alkyl group, provided that at least one of R^{1c}, R²c, R^{3c}, and R^{4c} is —O-R^{12c}, and n3 is an integer of 4 to 20.

23. The compound according to claim 22, wherein R^{1c} and R^{3c} are each independently —O-R^{12c}, or R^{2c} and R^{4c} are each independently —O-R^{12c}.

24. The compound according to claim 22, wherein n3 is an integer of 4 to 6.

25. The compound according to claim 22, wherein R^{1c} and R^{3c} are each —O-R^{12c}, and R^{2c} and R^{4c} are each a hydrogen atom, or R^{2c} and R^{4c} are each —O-R^{12c}, and R^{1c} and R^{3c} is each a hydrogen atom, R^{12c} is a C₁₇₋₃₀ alkyl group, and n3 is an integer of 4 to 6.

26. The compound according to claim 22, wherein when the compound is formed into a film, a static contact angle of the film with water is 80° or more.

27. A compound represented by Formula (1D) below: 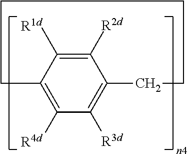
wherein R^{1d} is each independently at each occurrence a hydrogen atom or an organic group, R^{2d} is each independently at each occurrence a hydrogen atom or an organic group, R^{3d} is each independently at each occurrence a hydrogen atom or an organic group, and R^{4d} is each independently at each occurrence a hydrogen atom or an organic group, the organic group is each independently —O-R^{12d}, R¹²d is C₁₇₋₃₀ alkyl group, provided that at least one of R^{1d}, R^{2d}, R^{3d}, and R^{4d} is —O-R^{12d}, and R¹²³ has an SP value of 8.2 or more, and n4 is an integer of 4 to 20.

28. The compound according to claim 27, wherein R^{1d} and R^{3d} are each independently —O-R^{12d}, or R^{2d} and R^{4d} are each independently —O-R^{12d}.

29. The compound according to claim 27, wherein n4 is an integer of 4 to 6.

30. The compound according to claim 27, wherein the SP value of R^{12d} is 8.2 to 8.4.

31. The compound according to claim 27, wherein R^{1d} and R^{3d} are each —O-R^{12d} , and R^{2d} and R^{4d} are each a hydrogen atom, or R^{2d} and R^{4d} are each —O-R^{12d}, and R^{1d} and R^{3d} is each a hydrogen atom, R^{12d} is a C₁₇₋₃₀ alkyl group, and n4 is an integer of 4 to 6.

32. The compound according to claim 27, wherein when the compound is formed into a film, a static contact angle of the film with water is 80° or more.

33. A composition comprising at least one of the compound according to claim 1.

34. A formed article of the compound according to claim 1.

35. A formed article formed from the composition according to claim 33.

36. The formed article according to claim 34, wherein the formed article is a film.

37. A material comprising a substrate treated with the compound according to claim 1.
